(19) United States
(12) Patent Application Publication
(10) Pub. No.: US 2006/0178699 A1
(43) Pub. Date: Aug. 10, 2006

(54) Biopsy forceps

(75) Inventor: Vihar C. Surti, Winston-Salem, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 11/335,457

(22) Filed: Jan. 19, 2006

Related U.S. Application Data
(60) Provisional application No. 60/646,104, filed on Jan. 20, 2005.

(51) Int. Cl. A61B 17/28

(52) U.S. Cl. 606/205

(57) ABSTRACT

A biopsy forceps and method of using the biopsy forceps. The biopsy forceps includes a plurality of grasping members extending from an inner shaft. The plurality of grasping members are biased toward an open configuration. Sliding a sheath over the grasping members constrains the grasping members to a closed configuration. A method of performing a tissue biopsy is also disclosed.

This application claims the benefit of U.S. Provisional Application No. 60/646,104, filed Jan. 20, 2005.

### FIELD OF THE INVENTION

This invention generally relates to medical devices, and particularly to forceps used for obtaining biopsy samples.

### BACKGROUND

Physicians in many specialties commonly obtain biopsy samples from patients to determine the presence of tissue abnormalities, such as cancerous cells. Sometimes biopsies are taken without the need for an invasive procedure. For example, physicians can take skin biopsies to test for melanoma. In many cases, however, a physician must access a biopsy location inside a patient's abdominal cavity, thoracic cavity, or gastrointestinal system. For such procedures, physicians often use an endoscope to avoid more traumatic open surgery. Modern endoscopes are long, flexible instruments having a viewing system and a working channel through which a biopsy forceps can be passed.

Common endoscopic biopsy forceps are formed from a long shaft that extends between a proximal end and a distal end. The proximal end includes an actuator mechanism that a physician uses to control a small pair of biopsy jaws. The jaws are located at the distal end of the biopsy forceps, and are provided with teeth to cut, shear, or tear away tissue samples. For biopsy forceps that are used through the working channel of an endoscope, the shaft of the biopsy forceps is longer than the endoscope so that the biopsy forceps jaws can extend out of the distal end of the endoscope and reach the target tissue. Shorter biopsy forceps are used to take biopsies from locations where introduction of the biopsy forceps through an endoscope is unnecessary.

Conventional biopsy forceps, however, have a number of drawbacks. For example, the actuator and jaw mechanisms are formed from numerous and miniscule components that require manual assembly. The manufacture of biopsy forceps is therefore expensive, difficult, and time consuming. There is thus a need for a biopsy forceps that resolves or improves upon any of these drawbacks.

### SUMMARY OF THE INVENTION

Accordingly, it is an object of the present invention to provide a medical device having features that resolve or improve upon one or more of the above-described drawbacks.

According to one aspect of the present invention, the foregoing object is obtained by providing a biopsy forceps having a sheath and an inner shaft slidably disposed within the sheath. The shaft has a longitudinal axis defined therethrough and a plurality of grasping members that are movable between an open configuration and a closed configuration. At least one of the plurality of grasping members is biased outwardly from the longitudinal axis when in the open configuration. At least one of the plurality of grasping members is unrestricted by the sheath when in the open configuration and is constrained by the sheath when in the closed configuration. The plurality of grasping members can be formed so that when they are in a closed configuration, they form a receptacle for retaining one or more biopsy samples. One or more of the grasping members may further be provided with a cutting edge to more easily remove a tissue sample.

According to another aspect of the present invention the shaft may be connectable to an electrocautery device. As a result, the shaft may be energized by the electrocautery device to electrosurgically cut the tissue.

According to another aspect of the present invention, a method of performing a tissue biopsy is provided. The biopsy forceps can be introduced into a patient such that the grasping members are adjacent a target tissue. The sheath is then actuated, thereby closing the grasping members around the tissue. Once the grasping members are closed around the tissue, the shaft can be retracted to remove a portion of the target tissue. This procedure can be repeated to take multiple tissue biopsies.

### BRIEF DESCRIPTION OF THE DRAWINGS

Embodiments of the present invention will now be described by way of example with reference to the accompanying drawings (not to scale), in which:

FIG. 1 is a perspective side view of a biopsy forceps head according to an embodiment of the present invention;

FIG. 2 is a side view of a biopsy forceps head and a handle;

FIG. 3A is a cross-sectional view of a biopsy forceps head according to an embodiment of the present invention showing an open configuration taken along line 3-3 of FIG. 2;

FIG. 3B is an end view of the embodiment shown in FIG. 3A in a closed configuration having rectangularly shaped edges;

FIG. 3C is an end view of an alternative embodiment of the embodiment shown in FIG. 3A in a closed configuration having triangularly shaped edges;

FIG. 4 is a flow-chart of a method of using a biopsy forceps according to one embodiment of the present invention;

FIG. 5 is a side view of a biopsy forceps according to an embodiment of the present invention;

FIG. 6 is a cross-sectional front view of a biopsy forceps head according to an embodiment of the present invention;

FIG. 7 is an end view of a biopsy forceps head according to an embodiment of the present invention;

FIG. 8A is an end view of an embodiment in the closed configuration having two grasping members with rectangularly shaped edges;

FIG. 8A is an end view of an embodiment in the closed configuration having two grasping members with curved edges; and

FIG. 8A is an end view of an embodiment in the closed configuration having two grasping members with triangularly shaped edges.

### DETAILED DESCRIPTION

The invention is described with reference to the drawings in which like elements are referred to by like numerals. The relationship and functioning of the various elements of this invention are better understood by the following detailed description. However, the embodiments of this invention as described below are by way of example only, and the invention is not limited to the embodiments illustrated in the drawings. It should also be understood that the drawings are not to scale and in certain instances details that are not necessary for an understanding of the present invention have been omitted, such as conventional details of fabrication and assembly. Moreover, it should be noted that the invention described herein includes methodologies that have a wide variety of applications.

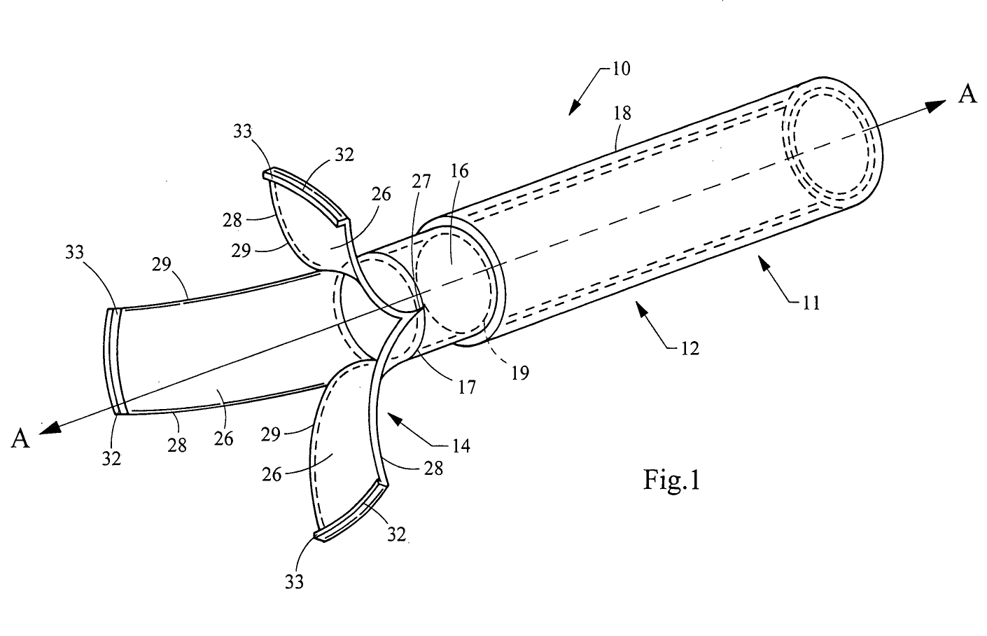

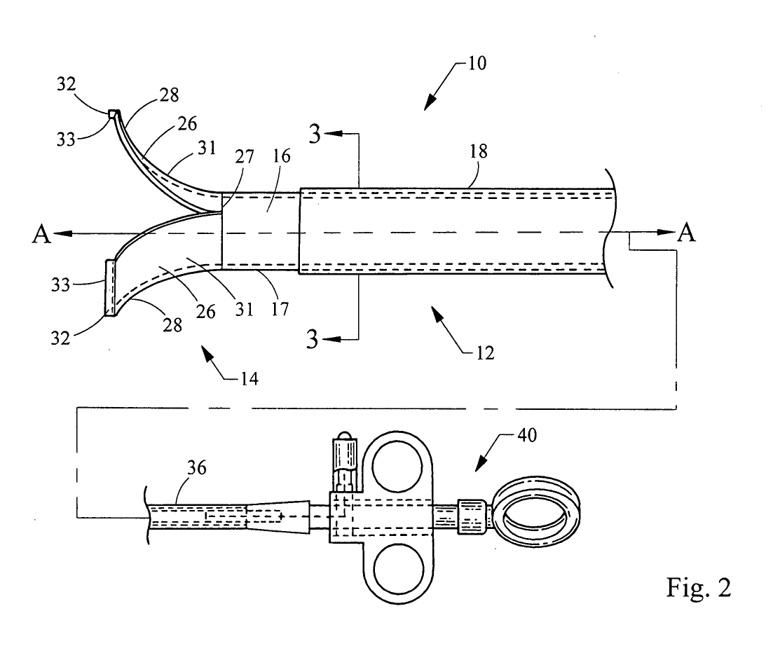

Referring to the drawings, FIGS. 1-3 depict an illustrative embodiment of the present invention. Generally, a medical device 10 is provided to take tissue samples for medical analysis. As illustrated in FIG. 1, the medical device 10 includes a catheter 11 having a distal end 12. The distal end 12 includes an inner shaft 16 and grasping members 26 extending from a distal end 17 of the shaft 16. A longitudinal axis A is defined through the shaft 16 as shown in FIGS. 1 and 2. The grasping members 26 are biased outwardly relative to the longitudinal axis A in an open configuration. The distal end 12 further includes an outer sheath 18 having a lumen 19 defined therein. In general, the shaft 16 is slidably received in the sheath 18 (i.e., within the lumen 19). The shaft 16 may be slidable relative to the sheath 18 so that the shaft 16 may be retracted at least partially into the sheath 18 to cam the grasping members 26 into a closed, contracted configuration. A conventional handle 40 (shown in FIG. 2) may be operably connected to a proximal end 36 of the catheter 11. The handle 40 may be used to control the movement of the shaft 16 relative to the sheath 18 and to control the movement of the grasping members 26 between the open configuration where the grasping members 26 are biased outwardly and the closed configuration where the grasping members 26 are cammed together.

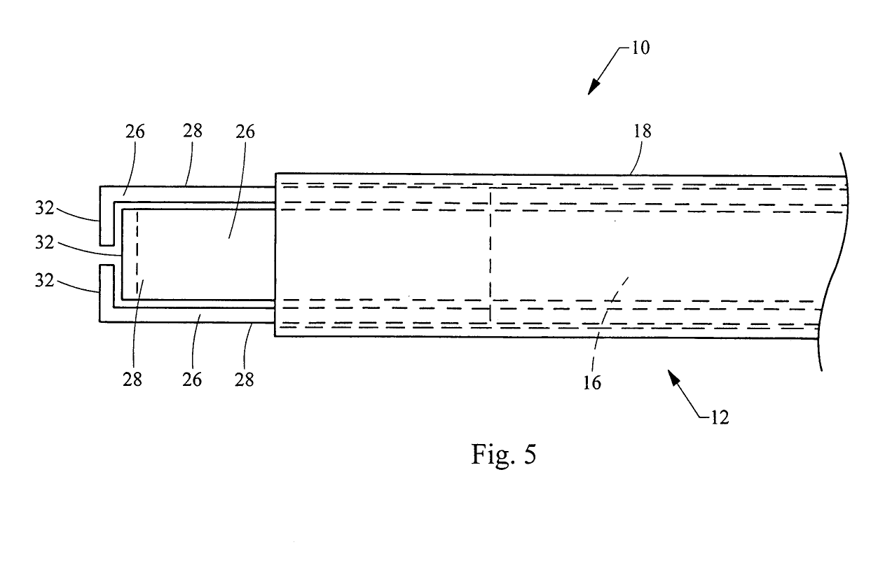

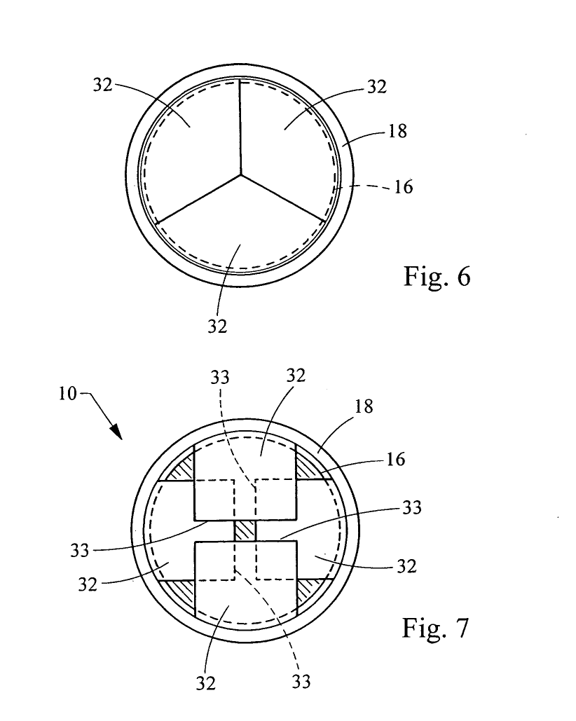

As illustrated in FIG. 1, the distal end 12 of the catheter 11 includes the inner shaft 16, grasping members 26 and the sheath 18. In some embodiments, three grasping members 26 may extend from the shaft 16, although two, three, four, five or more grasping members 26 are possible, as will be understood by one skilled in the art. The grasping members 26 include a proximal portion 27 connected to the distal portion 17 of the shaft 16 and a distal portion 28 extending distally. The grasping members 26 are shown having a curvilinear profile in the open configuration where the distal portion 28 is biased away from the longitudinal axis A (FIG. 1) and a substantially straight profile in the closed configuration (FIG. 5). Alternative profiles for the grasping members are also possible including bent profiles and the like. In some embodiments, the grasping members 26 may extend from the distal portion 17 of the shaft 16 and be formed by unitary construction with the shaft 16 from a single elongate member such as a tube described below. In some embodiments, the grasping members 26 and the shaft 16 may be formed from different components and connected together. For example, the proximal portions 27 of the grasping members 26 formed separately and may be connected to the shaft 16 at the distal portion 17 of the shaft 16, extending at an angle outward from the longitudinal axis A. In these embodiments, the grasping members 26 may have a straight profile or a curvilinear or bent profile and the like. The grasping members 26 include an outer surface 31 that can be seen in FIG. 2.

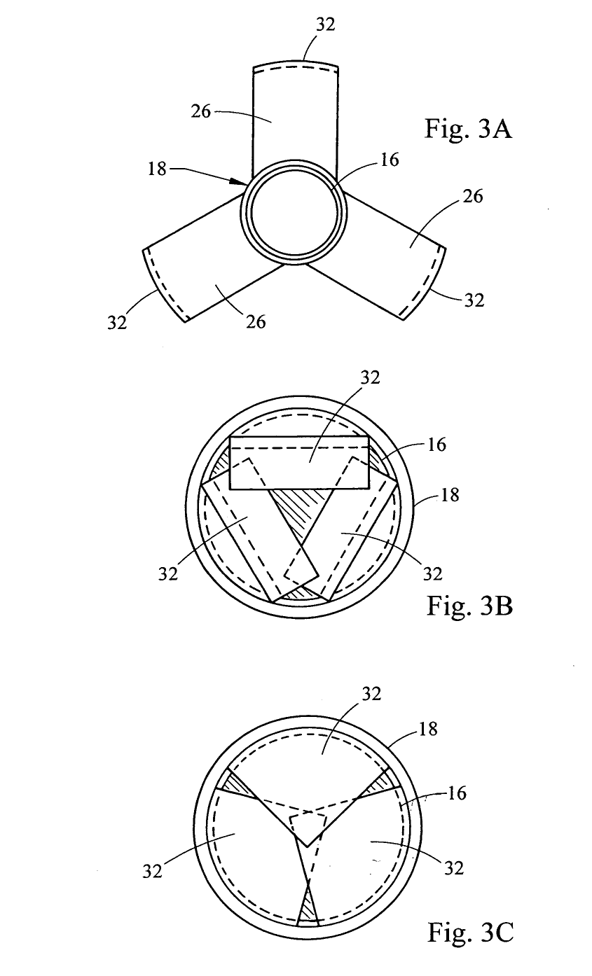

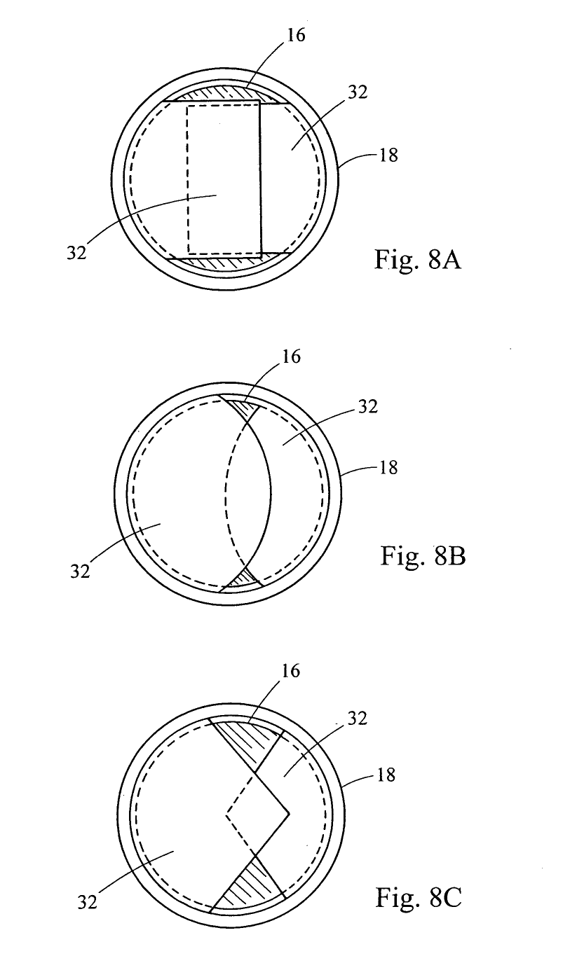

One or more of the grasping members 26 may be provided with a distal edge 32. In some embodiments, the distal edge 32 may be bent inwardly relative to the grasping member 26 and toward the longitudinal axis A, as shown in FIG. 2. The distal edge 32 may be bent at a 90° angle with respect to the grasping member 26. The distal edge 32 may be adapted for shearing, grasping, tearing, or cutting tissue. The edge 32 may further include a blade portion 33 having a cutting surface. The edge 32 and blade portion 33 may be formed in any shape and configuration, including, but not limited to, a single blade or cutting surface, a crenate tooth configuration, straight, angular or curved. The distal edge 32, the blade 33, or both may be shaped to fit together so that the edges 32 or the blades 33 meet together at the distal portion 28 in the closed configuration. FIGS. 3A-C illustrate an embodiment having three grasping members. FIG. 3A shows a cross-sectional view of the open configuration. FIG. 3B shows an end view having distal edges 32 with a rectangularly shaped portions that overlap in the closed configuration. FIG. 3C shows an alternatively shaped distal portion 28 having triangularly shaped distal edges 32 that overlap in the closed configuration. The embodiments shown in FIGS. 3B and 3C may also include the blade 33 on the distal edge 32. End views for alternative embodiments having two grasping members with alternatively shaped distal edges 32 are shown in FIGS. 8A-8C.

The distal edges 32 may be formed by removing material from the distal edge 32 to appropriately size and shape the edge 32 for fitting together, for example when the distal edge 32 is formed by bending a portion of the distal portion 28 of the grasping member 26 toward the longitudinal axis A. In embodiments having the blade 33, the blade 33 may be sized and shaped to fit together similarly to the distal edge 32. Alternatively, the distal edge 32 or the blade 33 or both may be formed by adding material to the distal portion 28 of the grasping members 26 in the desired size and shape.

In some embodiments, the distal edges 32 may be sized and shaped to overlap each other as shown in FIGS. 5 and 7. For example, as shown in FIG. 7 where the device 10 includes four grasping members 26, the opposite pairs of distal edges 32 may meet together with one pair extending further distally than the other pair so that the pairs overlap. In some embodiments, each distal edge 32 or blade 33 may overlap in the closed configuration, for example where three grasping members 26 are included with the device 10, the distal edges 32 may be triangularly shaped and overlap each other to form a generally triangularly shaped end of the receptacle, shown in end view in the closed configuration of FIG. 3B. Any shape and size may be used to form the edge 32 and the blade 33 so that in the closed configuration, the sample may be held within the chamber formed by the grasping members 26 and the edges 32 that meet or overlap in the closed configuration. The edges 32 may be blunt or may include blades 33 to remove the tissue sample from the patient.

In some embodiments, the grasping members 26 may be curved around the longitudinal axis A to form a generally annular profile similar to shaft 16 when the shaft 16 is a cylindrically shaped cross section. The shaft 16 and the sheath 18 may also have alternatively shaped cross-sectional shapes, including polygonal and oval, and the like. In some embodiments, the distal portion 28 of the grasping members 26 may be flattened with the distal edges 32 being rectangularly shaped as shown in FIG. 2. In some embodiments, the grasping members 26 may be relatively wide. The grasping members 26 may be used to capture a tissue sample within a chamber or receptacle formed by the grasping members 26 when the grasping members 26 are cammed together in the closed configuration. Longitudinal edges 29 of the grasping members 26 may be sized and shaped such that the longitudinal edges 29 of adjacent grasping members meet or are in proximity to form the chamber to hold the tissue sample when the device 10 is in the closed configuration. An example of the closed configuration is shown in FIG. 5. The configurations described above may allow the edges 32 to firmly grasp and cut or tear the tissue to be biopsied and may prevent the tissue sample from dislodging from the device 10.

In some embodiments the grasping members 26 and the shaft 16 may be formed from resilient materials known to one of skill in the art. Any elastic material that can retain bending stresses and resiliently return to its preformed shape may be used. In some embodiments, metal may be used to form the device 10 or components thereof. Exemplary metals include stainless steel or an alloy having superelastic properties such as nitinol (NiTi). The shaft 16 and the grasping members 26 may be formed from a single piece of stainless steel tubing. A conventional programmable laser cutter can be programmed to laser-cut the tubing into the desired configuration. The laser cutter may be programmed to cut the desired shape repeatedly from a single length of tubing. The laser cutter may similarly be programmed to cut the shaft 16 to form any number of grasping members 26 (e.g., two, three, four, five, six, or more grasping members). Alternatively, the grasping members 26 may be welded or otherwise attached to the shaft 16 using techniques known to one skilled in the art. The grasping members 26 may be equally sized and shaped, or the grasping members 26 may be differently sized and shaped, for example, alternating between wider and narrower grasping members 26 or longer and shorter pairs of grasping members 26. The laser cutter may also be used to form the distal edges 32 and the blades 33 into any desired size and shape, for example, by removing a portion of material of the edges 32.

As illustrated in FIGS. 1-3, the sheath 18 may be slidably disposed over a portion of the shaft 16 to constrain the grasping members 26. The sheath 18 may slide relative to the shaft 16 to engage at least a portion of the outer surface 31 of the grasping members 26 to constrain the grasping members 26 in the closed configuration. In an exemplary embodiment, the sheath 18 may slide a distance between about 2 millimeters and 10 millimeters relative to the shaft 16, although a person of ordinary skill could alter the distance that the sheath 18 slides relative to the shaft 16. As the shaft 16 is retracted into or slides into the sheath 18—or alternatively as the sheath 18 slides forward over the shaft 16—the sheath 18 may cam the grasping members 26 into a closed configuration and constrain the grasping members 26. In some embodiments, the shaft 16, the sheath 18 or both may include a portion having a thin layer of lubricious material, such as polytetrafluoroethylene (PTFE) on surfaces that may contact each other, including the outer surface 31 of the grasping members 26. Sliding the sheath 18 in relation to the shaft 16 causes the grasping members 26 and the edges 32 to firmly grasp and cut, shear, or tear the tissue to be biopsied. The closed configuration with the sheath 18 slidably disposed over at least a portion of the grasping members 26 in the closed configuration may also allow for a narrower configuration for easy of delivery through a medical device, such as the working channel of an endoscope. The overall size and shape of the device 10 will depend on the location in which the device 10 will be used.

Operation of the biopsy forceps device 10 may be performed by any means known to one skilled in the art. For example, remote operation of the biopsy forceps device 10 may be controlled via a handle 40 at the proximal end 36 (FIG. 2, showing an enlarged distal portion 12). As will become apparent to a person of ordinary skill, a wide variety of handle mechanisms could be used with the present invention. The handle 40 may be a thumb ring, a scissors-type handle, a pin vise, or any other conventional handle suitable for moving a sheath relative to a control wire or shaft. The handle 40 may also be connected to a control wire which is connected to the shaft 16 or the sheath 18. In general, the handle 40 is used to actuate the control wire, which in turn controls the movement of one of the shaft 16 or sheath 18 in relation to the other. In addition, the handle 40 may be used to maneuver the biopsy forceps device 10.

An electrical connector may be provided to energize the shaft 16 and grasping members 26 of the device 10. The electrical connector may conveniently form a male plug, which receives an electrical cord (sometimes called an ‘active cord’). The electrical cord is connectable to a standard electrosurgical generator, such as those manufactured by Valleylab, Inc. (Boulder, Colo.). In use, a physician, via the generator, controls whether current is applied to the device 10, typically using a foot pedal to electrify the control wire and ablate tissue coming in contact with the stem, grasping members, or cutting edges. This allows a physician to cut or cauterize bleeding tissues with the shaft 16, grasping members 26, or cutting edges 32. The sheath 18 may be coated with insulating material, such as plastic or rubber, in some embodiments, as will be understood by one skilled in the art.

In some embodiments of the present invention, the biopsy forceps device 10 may be operably connected to an infusion source or a suction source. For example, in embodiments a suction device, such as a vacuum or a syringe may be connected to the shaft 16 to assist in tissue removal or general fluid removal around the biopsy site. The suction source may also be used to pull the biopsy sample back into the shaft 16 for removal or for taking multiple biopsy samples. Alternatively or in addition, the biopsy forceps device 10 may be operably connected to an infusion source such as a syringe or a pump to provide fluid to the biopsy site through the shaft 16. For example, saline, dye or medication may be infused through the shaft 16 to the biopsy site. Alternative lumens may be provided in the biopsy device 10 in addition to the shaft 16 to provide infusion or suction as needed. Any infusion device or suction source known to one skilled in the art may be operably connected to the biopsy device 10.

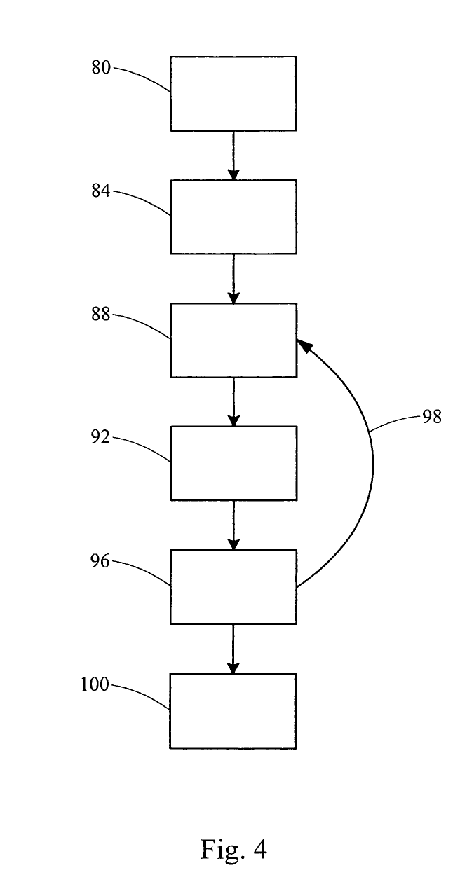

FIG. 4 illustrates one method of utilizing the present invention to remove a biopsy sample from a target tissue. As illustrated in step 80, the target tissue is located, for example by using an endoscope. Once the target tissue is located, the biopsy forceps device 10 may be delivered to the target tissue, for example, by introducing the biopsy forceps device 10 through the working channel of the endoscope as illustrated in step 84. Alternatively the device 10 may be delivered to the tissue at the same time the tissue is being located. When the target tissue is located, the device 10 may be advanced toward the target biopsy tissue until the cutting edges 32 and/or the grasping members 26 are in contact with the target biopsy tissue, as shown in step 88. At this point in the procedure, a physician may manipulate the handle so as to slide either the shaft 16 or the sheath 18 to manipulate the grasping members 26 into position and then cam the grasping members 26 at least partially into the sheath 18 to move the grasping members 26 into the closed configuration, thereby grasping the targeted biopsy tissue, as shown in step 92. Thereafter, the physician may energize the grasping members 26 and shaft 16 with the energizing source, or simply tear, shear, or cut off a biopsy sample by pulling the device 10 away from the tissue, as shown in step 96. Optionally, the physician may take additional samples of the target tissue by repeating steps 88-96, as illustrated by step 98. Once the desired number of biopsy samples is taken, the physician can withdraw the device 10 and retrieve the biopsy sample for analysis, as shown in step 100.

Any other undisclosed or incidental details of the construction or composition of the various elements of the disclosed embodiment of the present invention are not believed to be critical to the achievement of the advantages of the present invention, so long as the elements possess the attributes needed for them to perform as disclosed. The selection of these and other details of construction are believed to be well within the ability of one of even rudimentary skills in this area, in view of the present disclosure. Illustrative embodiments of the present invention have been described in considerable detail for the purpose of disclosing a practical, operative structure whereby the invention may be practiced advantageously. The designs described herein are intended to be exemplary only. The novel characteristics of the invention may be incorporated in other structural forms without departing from the spirit and scope of the invention. Unless otherwise indicated, all ordinary words and terms used herein shall take their customary meaning as defined in The New Shorter Oxford English Dictionary, 1993 edition. All technical terms shall take on their customary meaning as established by the appropriate technical discipline utilized by those normally skilled in that particular art area. All medical terms shall take their meaning as defined by Stedman's Medical Dictionary, 27th edition.

What is claimed is:

1. A biopsy forceps comprising:
a sheath; and
an inner shaft slidably disposed within the sheath and having a longitudinal axis defined therethrough, the shaft comprising a plurality of grasping members that are movable between an open configuration and a closed configuration, wherein at least one of the plurality of grasping members is biased outwardly from the longitudinal axis when in the open configuration,
wherein the at least one of the plurality of grasping members is unrestricted by the sheath when in the open configuration and is constrained by the sheath when in the closed configuration.

2. The biopsy forceps of claim 1, wherein one or more of the grasping members includes a cutting edge.

3. The biopsy forceps of claim 2, wherein the cutting edge comprises a blade portion.

4. The biopsy forceps of claim 1, wherein the plurality of grasping members form a biopsy receptacle when in the closed configuration.

5. The biopsy forceps of claim 1, wherein the shaft is formed from stainless steel.

6. The biopsy forceps of claim 2, wherein the cutting edge is curvilinear.

7. The biopsy forceps of claim 2, wherein the cutting edge comprises a jagged edge configured to tear tissue from a biopsy site.

8. The biopsy forceps of claim 2, wherein the cutting edge is bent inward toward the longitudinal axis.

9. The biopsy forceps of claim 1, wherein the shaft and the grasping members are formed by unitary construction from the same material.

10. A biopsy forceps comprising:
a sheath having a lumen formed therein;
a shaft slidably received within the lumen, the shaft having a proximal portion and a distal portion and a longitudinal axis defined through the shaft;
a plurality of grasping members extending distally from the distal portion of the shaft and at least on of the plurality of grasping members extending outwardly from the longitudinal axis;
wherein the sheath is slidable relative to the shaft to constrain the plurality of grasping members in a closed configuration.

11. The biopsy forceps of claim 10, wherein one or more of the grasping members includes a cutting edge.

12. The biopsy forceps of claim 11, wherein the cutting edge comprises a blade portion.

13. The biopsy forceps of claim 10, wherein the sheath has a cylindrically shaped cross section.

14. The biopsy forceps of claim 10, wherein at least one of the plurality of grasping members has a profile that is substantially parallel to the longitudinal axis in the closed configuration and a profile that is curvilinear in the open configuration

15. The biopsy forceps of claim 10, wherein the sheath engages an outer surface of the plurality of grasping members to constrain the grasping members.

16. The biopsy forceps of claim 10, wherein each one of the plurality of grasping members includes a distal portion and a proximal portion and the proximal portion is connected to the shaft.

17. The biopsy forceps of claim 10, wherein a portion of the shaft is connectable to an electrocautery source to electrosurgically cut tissue.

18. A method of performing a tissue biopsy, the method comprising:
a) providing a biopsy forceps, comprising:
a shaft defining a proximal portion, a distal portion, and a longitudinal axis, the shaft comprising a plurality of grasping members, each of the plurality of resilient grasping members being biased away from the longitudinal axis; and
a sheath slidable between a closed configuration in which the sheath constrains the plurality of grasping members and an open configuration in which the plurality of grasping members are unrestricted by the sheath;
b) positioning a portion of the biopsy forceps adjacent to the tissue;
c) sliding the sheath to constrain the grasping members in the closed configuration around the tissue; and
d) retracting the shaft and removing a tissue biopsy.

19. The method of claim 18 further comprising providing at least one blade operable connected to at least one of the grasping members for cutting the tissue.

20. The method of claim 18 further comprising providing a cutting edge one or more of the plurality of grasping members.
